(19) United States
(12) Patent Application Publication
(10) Pub. No.: US 2024/0203588 A1
(43) Pub. Date: Jun. 20, 2024

(54) DIAGNOSTIC DISEASE PREDICTION AND THERAPEUTIC PLAN RECOMMENDATION SYSTEM THROUGH ARTIFICIAL INTELLIGENCE QUESTIONNAIRE ANALYSIS AND METHOD THEREOF

(71) Applicant: BePlus Healthcare Inc., Gangwon-do (KR)

(72) Inventors: Hoon Jae CHUNG, Seoul (KR); Ki Joon HUGH, Busan (KR); Jae Young KIM, Seoul (KR); Jee Hong KIM, Seoul (KR); Woo Taek LIM, Seoul (KR)

(21) Appl. No.: 18/203,729

(22) Filed: May 31, 2023

(30) Foreign Application Priority Data
Dec. 19, 2022 (KR) 10-2022-0178504

(51) Int. Cl. G16H 50/20; G16H 10/20; G16H 10/40; G16H 10/60

(52) U.S. Cl. CPC G16H 50/20; G16H 10/20; G16H 10/40; G16H 10/60

(57) ABSTRACT

A diagnostic disease prediction system according to an embodiment is based on a remote interactive questionnaire to provide the patient and the medical personnel with a disease prediction result, thereby helping a medical consumer correctly identify his/her condition and select an appropriate medical department and helping medical personnel quickly select a patient in emergency. The system includes a database (DB) storing a questionnaire scenario, a questionnaire analysis unit analyzing a questionnaire answer acquired from a patient by using an analysis artificial intelligence, a disease prediction unit generating a disease prediction result by analyzing a patient symptom through a questionnaire result systematized using a diagnosis artificial intelligence, a test selection unit selecting a necessary test item based on the disease prediction result, and a communication unit delivering the disease prediction result and the selected necessary test item to the patient or a medical institution.

### CROSS REFERENCE TO RELATED APPLICATIONS AND CLAIM OF PRIORITY

This application claims benefit of priority to Korean Patent Application No. 10-2022-0178504, filed on Dec. 19, 2022, in the Korean Intellectual Property Office, the disclosure of which is incorporated herein by reference in its entirety.

### BACKGROUND

### 1. Field

The present disclosure relates to a diagnostic disease prediction and therapeutic plan recommendation system and a method thereof, and more particularly, to a diagnostic disease prediction and therapeutic plan recommendation system through an artificial intelligence questionnaire analysis and a method thereof.

### 2. Description of the Related Art

Questionnaires indicate a process of acquiring medical data for a patient, such as the main symptom, symptom characteristic, occurrence time, associated symptom, past history and family history of the patient, in the form of questions and answers. Medical personnel may determine urgency of the symptom complained of by the patient through the questionnaire, identify a disease requiring a differential diagnosis, and establish a plan for future diagnosis and therapy. However, the data acquired through the questionnaire in a clinical field so far is unstructured medical data freely recorded in an arbitrary format by the medical personnel such as doctors and nurses. Accordingly, there are many difficulties in processing such data into meaningful data and using the same for a statistical analysis or the like.

Meanwhile, importance of various medical data necessary for the development of an artificial intelligence has emerged as attempts to introduce the artificial intelligence to a medical field are increased to provide the patient with more optimized medical care. However, the questionnaire data are not properly utilized in practice although these data are important data playing a key role in early clinical determination because it takes a lot of time and cost to secure dedicated personnel for standardizing and refining the data for the data to be used for the development of the artificial intelligence.

In order to overcome a limitation of the unstructured data, attempts have been made to collect a questionnaire result in a structuralized form by providing standardized questionnaire through a medium such as a written or web page. However, such a questionnaire system often uses a method of requiring a short answer such as yes or no, and it is thus difficult to collect more detailed data from the patient.

Meanwhile, an attempt has been made to develop a questionnaire-based diagnostic application for inferring a disease that the patient is highly likely to have from the symptom complained of by the patient through the questionnaire or determining the severity of the disease. However, a conventional questionnaire-based diagnostic system based on a structuralized computer thinking form such as a tree-type algorithm may repeat similar questions multiple times or provide unnecessary questions, thus causing a user (or the patient) to feel strange and the diagnosis to be less accurate.

### SUMMARY

An aspect of the present disclosure is to provide a diagnostic disease prediction system based on a remote interactive questionnaire and thus provide the patient and the medical personnel with a disease prediction result, thereby helping a medical consumer correctly identify his/her condition and select an appropriate medical department and helping medical personnel quickly select a patient in emergency.

Another aspect of the present disclosure is to provide appropriate initial medical care ensured in a special environment such as a remote medical care or a disaster situation, where medical resources are extremely limited, by recommending necessary test and therapy in the future, management plan, or the like based on disease prediction through a questionnaire.

Another aspect of the present disclosure is to provide disease prediction with higher accuracy by recommending a future necessary test based on the disease prediction through questionnaire, and by reflecting a test result received through a linked medical device in a diagnosis process to correct a disease prediction result.

Another aspect of the present disclosure is to provide a diagnosis artificial intelligence with higher long-term accuracy by continuously supplementing the diagnosis artificial intelligence of the diagnostic disease prediction system through questionnaire by using a feedback based on data such as a test result and an electronic medical record (EMR).

Another aspect of the present disclosure is to provide medical data acquired through a questionnaire system with higher efficiency and interoperability by structuralizing and standardizing the components and results of questionnaire by the questionnaire system using ‘systematized nomenclature of medicine clinical terms (SNOMED-CT)’ which are international standard medical terms and ‘Korean standard terminology of medicine (KOSTOM)’ which is a Korean standard medical terminology developed by the Ministry of Health and Welfare in Korea.

In an aspect, a diagnostic disease prediction and therapeutic plan recommendation system through an artificial intelligence questionnaire analysis may include: a database (DB) storing a questionnaire scenario: a questionnaire analysis unit analyzing a questionnaire answer acquired from a patient by using an analysis artificial intelligence: a disease prediction unit generating a disease prediction result by analyzing a patient symptom through a questionnaire result systematized using a diagnosis artificial intelligence: a test selection unit selecting a necessary test item based on the disease prediction result; and a communication unit delivering the disease prediction result and the selected necessary test item to the patient or a medical institution.

The system may further include: a test result receiving unit linked with a medical device and receiving a test result of the necessary test item; a diagnosis determination unit analyzing the test result and reviewing the disease prediction result: and a diagnosis result correction unit generating a corrected disease prediction result by reflecting the test result when the disease prediction result includes an error.

The system may further include a diagnosis performance enhancement unit enhancing performances of the analysis artificial intelligence and the diagnosis artificial intelligence by using a performance-enhancement artificial intelligence to evaluate accuracies of the analysis artificial intelligence and the diagnosis artificial intelligence with reference to one or more of the test result and electronic medical record (EMR) data of the patient and provide continuous feedback.

The system may further include a disease prevention and management unit providing the patient with health coaching and health counseling based on the disease prediction result.

The system may further include a medical cost prediction unit predicting a possible medical cost based on the disease prediction result and the necessary test item, and providing the patient with the predicted cost.

In another aspect, a diagnostic disease prediction and therapeutic plan recommendation method through an artificial intelligence questionnaire analysis may include: systematizing questionnaire answers input by a patient by using an analysis artificial intelligence: generating a disease prediction result by using a diagnosis artificial intelligence to identify a patient condition based on a questionnaire result and predict a disease that the patient is highly likely to have: selecting a necessary test item based on the disease prediction result; and delivering the disease prediction result and the necessary test item to the patient or a medical institution.

The method may further include: receiving a test result of the necessary test item from a linked medical device; evaluating the disease prediction result by analyzing the test result: and generating a corrected disease prediction result by correcting the disease prediction result when the disease prediction result includes an error.

The method may further include enhancing accuracies of the analysis artificial intelligence and the diagnosis artificial intelligence by using a performance-enhancement artificial intelligence to evaluate performances of the analysis artificial intelligence and the diagnosis artificial intelligence with reference to one or more of the test result and electronic medical record (EMR) data of the patient and provide continuous feedback.

The method may further include providing the patient with health coaching and health counseling based on the disease prediction result.

The method may further include predicting a possible medical cost based on the disease prediction result and the necessary test item, and providing the patient with the predicted cost.

As set forth above, the system and the method according to the present disclosure may provide the questionnaire-based disease prediction with the higher accuracy by performing the necessary test by using the linked medical device and reflecting its result in the diagnosis to correct the disease prediction result.

The system and the method according to the present disclosure may provide the artificial intelligence with the continuously enhanced performance by providing the artificial intelligence with the data such as the final diagnosis result of the patient that is registered in the linked electronic medical record (EMR) as the feedback.

The system and the method according to the present disclosure may help the medical personnel to identify the detailed patient condition in the medical situation where many patients need to be treated in a limited time by providing the essential questionnaire before the treatment, and reduce the misdiagnoses occurring due to the insufficient data by securing the enhanced efficiency of the communication between the medical personnel and the patient.

The system and the method according to the present disclosure may help the medical personnel not to overlook the important or critical disease of the patient by selecting medically important questions and providing the same to the patient in advance.

The system and the method according to the present disclosure may provide the promoted efficient use of the limited medical resources in the special situation where the medical resources are extremely limited, such as the military medical care, the disaster situation, or the remote medical care, by providing the low-skilled medical personnel with the primary diagnosis and severity data of the patient and recommending the necessary test and therapy direction first.

The system and the method according to the present disclosure may provide the maximized interoperability and efficiency of the questionnaire data for the development of the artificial intelligence, the statistical research, or the like by loading the collected questionnaire results into the structuralized data based on the standard terms such as the SNOMED-CT.

### BRIEF DESCRIPTION OF THE DRAWINGS

FIG. 1 is a view for explaining a diagnostic disease prediction and therapeutic plan recommendation system through an artificial intelligence questionnaire analysis according to an embodiment of the present disclosure.

FIG. 2 is a view showing a configuration of the diagnostic disease prediction and therapeutic plan recommendation system through an artificial intelligence questionnaire analysis according to an embodiment of the present disclosure.

FIG. 3 is a view showing a method of enhancing performance of therapeutic plan recommendation and disease prediction according to an embodiment of the present disclosure.

FIG. 4 is a view for explaining a diagnostic disease prediction and therapeutic plan recommendation method through an artificial intelligence questionnaire analysis according to another embodiment of the present disclosure.

FIG. 5 is a view showing a method of correcting a disease prediction result by reflecting a test result according to another embodiment of the present disclosure.

### DETAILED DESCRIPTION OF THE EMBODIMENTS

Various advantages and features of the present disclosure and methods accomplishing the same are apparent from embodiments described below in detail with reference to the accompanying drawings.

However, the present disclosure is not limited to the embodiments described below, and may be implemented in various different forms.

These embodiments in the specification are provided only to make the present disclosure complete and allow those skilled in the art to which the present disclosure pertains to completely appreciate the scope of the present disclosure.

In addition, the present disclosure is defined by the scope of the claims.

Therefore, in some embodiments, well-known components, well-known operations, and well-known techniques are not described in detail in order to avoid ambiguous interpretation of the present disclosure.

In addition, like reference numerals throughout the specification denote like elements, and terms used (or referred to) in the specification are provided for describing the embodiments and are not intended to limit the present disclosure.

In the specification, a term of a singular number may include its plural number unless specifically indicated otherwise in the context, and components and operations referred to as being “included (or provided)” do not exclude the presence or addition of one or more other components and operations.

Unless defined otherwise, all terms (including technical and scientific terms) used in the specification have the same meaning as meanings commonly understood by those skilled in the art to which the present disclosure pertains.

In addition, terms generally used as defined in a dictionary are not to be interpreted as having ideal or excessively formal meanings unless clearly indicated otherwise.

Hereinafter, the embodiments of the present disclosure are described with reference to the accompanying drawings.

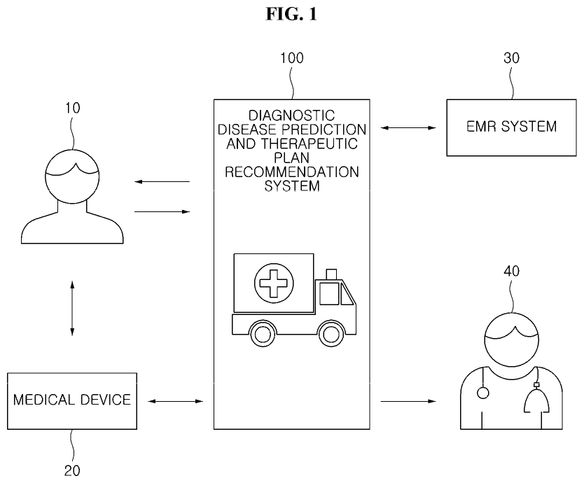

FIG. 1 is a view for explaining a diagnostic disease prediction and therapeutic plan recommendation system through an artificial intelligence questionnaire analysis according to an embodiment of the present disclosure.

A disease prediction and therapeutic plan recommendation system 100 through an artificial intelligence questionnaire analysis of the present disclosure may analyze a questionnaire answer acquired from a patient 10 to select a necessary test, perform a diagnosis based on a test result, and then deliver a diagnosis result to the patient 10 or medical personnel 40.

The disease prediction and therapeutic plan recommendation system 100 through an artificial intelligence questionnaire analysis of the present disclosure may be installed, for example, in a mobile hospital such as a vehicle, and may thus be used to quickly identify a patient condition and take action in an emergency situation where available medical resources are extremely limited.

Hereinafter, for convenience of explanation, the diagnostic disease prediction and therapeutic plan recommendation system 100 through an artificial intelligence questionnaire analysis is referred to as the diagnostic disease prediction and therapeutic plan recommendation system 100.

The diagnostic disease prediction and therapeutic plan recommendation system 100 may provide a disease prediction result by analyzing the questionnaire answer received from the patient 10 to thus select a series of diseases each having a high possibility, while selecting a test item to be further performed on the patient to increase accuracy of the diagnosis. The system 100 may then allow the necessary test to be performed by providing the patient or a medical institution with the selected test item. In addition, the system 100 may provide more accurate disease prediction by being linked with a surrounding medical device 20 to receive the test result of the patient, reflecting the same in a diagnosis process, and thus reviewing and correcting the accuracy of previous disease prediction.

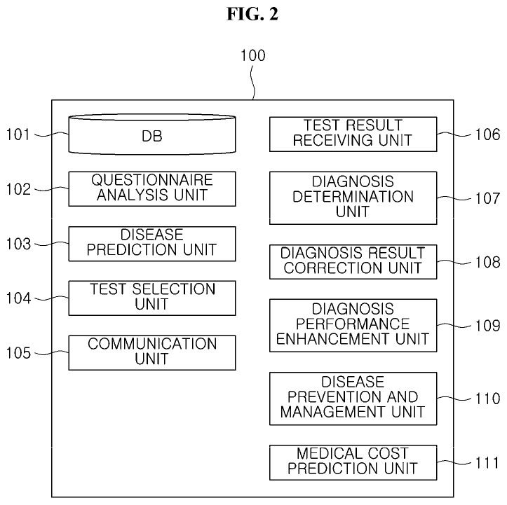

In addition, the system 100 may enhance accuracy of a questionnaire analysis artificial intelligence and a diagnosis artificial intelligence by evaluating accuracy of the disease prediction result with reference to one or more of the test result received from the medical device 20 and medical data of the patient received from an electronic medical record (EMR) system 30, and providing continuous feedback to the artificial intelligence. FIG. 2 is a view for showing a configuration of the diagnostic disease prediction and therapeutic plan recommendation system through an artificial intelligence questionnaire analysis according to an embodiment of the present disclosure.

The diagnostic disease prediction and therapeutic plan recommendation system 100 of the present disclosure may include a database (DB) 101, a questionnaire analysis unit 102, a disease prediction unit 103, a test selection unit 104, a communication unit 105, a test result receiving unit 106, a diagnosis determination unit 107, a diagnosis result correction unit 108, a diagnosis performance enhancement unit 109, a disease prevention and management unit 110, and a medical cost prediction unit 111.

The DB 101 may store a questionnaire scenario.

Here, the questionnaire scenario indicates a questionnaire set to identify a patient symptom. That is, the questionnaire scenario indicates a set including selected questionnaire essential for the diagnosis based on basic data of the patient and the symptom mainly complained of by the patient.

Here, each scenario may be established based on standardized terminology such as ‘Korean standard terminology of medicine (KOSTOM)’ and ‘systematized nomenclature of medicine clinical terms (SNOMED-CT)’. Accordingly, questionnaire data may be loaded to be easily exchanged with another database such as an electronic medical record (EMR).

The questionnaire analysis unit 102 may systematize the questionnaire answer acquired from the patient by using the artificial intelligence.

The questionnaire analysis unit 102 may classify and structuralize the questionnaire answers for each item input by the patient based on the items and attributes necessary for the diagnosis by using an analysis artificial intelligence, standardize a patient answer corresponding to each attribute based on standard medical terms such as the SNOMED-CT, and encode and store the standardized patient answer for the answer to be analyzed by the artificial intelligence.

The disease prediction unit 103 may generate the disease prediction result of a disease that the patient is highly likely to have by using the artificial intelligence based on a structuralized questionnaire result.

That is, the disease prediction unit 103 may select the disease that the patient is highly likely to have by synthesizing data on the symptom, past history, or the like of the patient from the patient questionnaire data previously structuralized by using the diagnosis artificial intelligence.

The test selection unit 104 may select a necessary test item based on the disease prediction result.

That is, when the disease prediction result of the patient is generated, the test selection unit 104 may select the necessary test item based on the result. For example, when the result is generated that the patient is highly likely to have pneumonia based on the questionnaire result, the test selection unit 104 may select a test (e.g., auscultation, chest X-ray test, or blood inflammation level test) necessary for differential diagnosis of pneumonia based thereon.

The communication unit 105 may deliver the disease prediction result and the selected test item to the patient or the medical institution.

Here, the medical institution may include a facility such as a hospital that stores the medical data by using the electronic medical record (EMR) system 30 or the like, and the medical personnel 40 who treat the patient.

The test result receiving unit 106 may be linked with the medical device 20 and receive results of the various tests performed on the patient 10.

The diagnosis determination unit 107 may re-evaluate the disease prediction result based on the received test result.

That is, the diagnosis determination unit 107 may evaluate whether the received test result matches the previously-provided disease prediction result, determine that the previous disease prediction result includes an error when the disease prediction result does not match the test result, and perform work to re-execute the diagnosis by reflecting the test result.

The diagnosis result correction unit 108 may generate a corrected disease prediction result by correcting the disease prediction result when the disease prediction result is determined as including the error.

That is, the diagnosis result correction unit 108 may re-configure the disease prediction result by reflecting the test result in the diagnosis process when the disease prediction result provided prior to the test and the test result do not match with each other.

Here, the diagnosis result correction unit 108 may newly select only an additionally necessary questionnaire from the previously-provided questionnaire scenario in addition to the test result to increase the accuracy of the disease prediction result, and provide the same to the patient. The diagnosis result correction unit 108 may then generate the corrected disease prediction result by transmitting the newly received answer and test result to the above-described questionnaire analysis unit 102 and disease prediction unit 103 to thus re-execute the analysis and diagnosis processes.

The diagnosis performance enhancement unit 109 may enhance performances of analysis and diagnosis algorithms by using a performance-enhancement artificial intelligence to evaluate whether the analysis and the diagnosis are accurate with reference to one or more of the test result and the electronic medical record (EMR) data of the patient, and provide the continuous feedback.

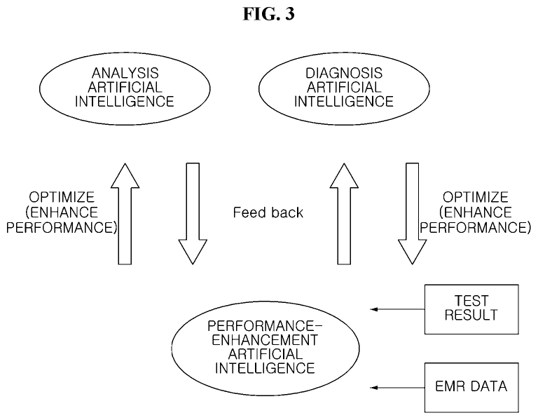

A detailed description of the diagnosis performance enhancement unit 109 is provided with reference to FIG. 3. The disease prevention and management unit 110 and the medical cost prediction unit 111 are additional components of the test and diagnosis prediction system 100 of the present disclosure. The disease prevention and management unit 110 may provide the patient 10 with health coaching and health counseling to form a healthy lifestyle of the patient based on the disease prediction result, if necessary. The medical cost prediction unit 111 may predict a possible medical cost based on the disease prediction result and the necessary test item, and provide the patient 10 with the predicted cost.

FIG. 3 is a view showing a method of enhancing the performances of the test and the disease prediction according to an embodiment of the present disclosure.

The diagnosis performance enhancement unit 109 may enhance the performances of the analysis artificial intelligence and the diagnosis artificial intelligence by using the performance-enhancement artificial intelligence.

That is, the performance-enhancement artificial intelligence may analyze completeness of the questionnaire data provided by the analysis artificial intelligence based on the test result from the linked medical device and the electronic medical record (EMR) data of the linked medical institution, provide an indicator for evaluating the accuracy of the disease prediction made by the diagnosis artificial intelligence based on data such as a final diagnosis, and analyze the performances of the analysis artificial intelligence and the diagnosis artificial intelligence to enable these artificial intelligences to be periodically optimized.

Here, the analysis artificial intelligence may compare the questionnaire result input by the patient with the patient data recorded in the electronic medical record (EMR) or the like to evaluate the accuracy of the questionnaire result input by the patient, and then reflect the evaluated accuracy in the questionnaire scenario to enhance performance of the questionnaire scenario.

Here, the diagnosis artificial intelligence may analyze the patient data and the questionnaire result to compare the data such as the generated disease prediction result, the test result acquired from the medical device, and the final diagnosis recorded in the electronic medical record (EMR) with one another, thereby evaluating the accuracy of the disease prediction result and utilizing the same to enhance its performance.

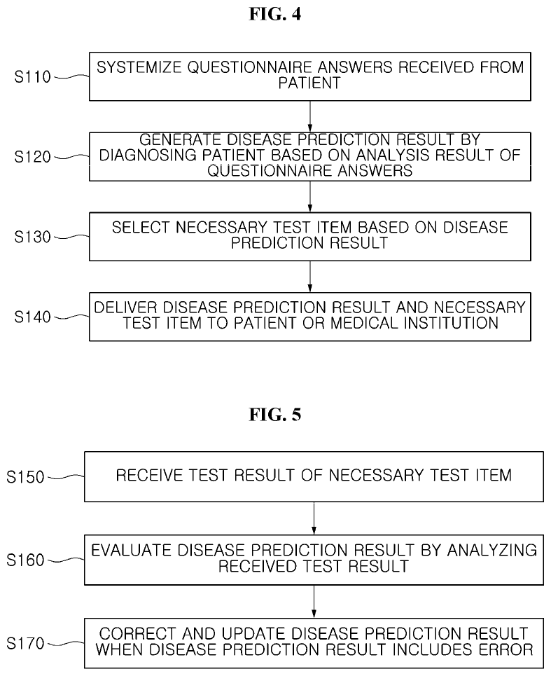

FIG. 4 is a view for explaining a diagnostic disease prediction and therapeutic plan recommendation method through an artificial intelligence questionnaire analysis according to another embodiment of the present disclosure.

The method may include systemizing questionnaire answers received from a patient for the answer to be analyzed by an artificial intelligence (S110).

The diagnostic disease prediction and therapeutic plan recommendation system 100 of the present disclosure may structuralize, standardize, and encode the questionnaire answer received through an analysis artificial intelligence to refine the same into data that may be utilized for a diagnosis.

Structuralizing indicates re-configuring the questionnaire answer by classifying a patient answer for each questionnaire item based on the items and attributes necessary for the diagnosis.

Standardizing indicates re-configuring each classified answer by replacing the answer with a combination of terms having the same meaning in standard terms such as ‘systematized nomenclature of medicine clinical terms (SNOMED-CT)’.

Encoding indicates that a code such as the SNOMED-CT or Korean classification of diseases (KCD) is additionally assigned to a standardized questionnaire result.

The method may include generating a disease prediction result by selecting a disease that the patient is highly likely to have based on the analyzed questionnaire result (S120).

The diagnostic disease prediction and therapeutic plan recommendation system 100 of the present disclosure may select the disease that the patient is highly likely to have by synthesizing health data such as the symptom, past history, or the like of the patient from the questionnaire result refined using a diagnosis artificial intelligence, and thus generate the disease prediction result.

The method may include selecting a necessary test item based on the disease prediction result (S130).

The diagnostic disease prediction and therapeutic plan recommendation system 100 of the present disclosure may identify a patient condition through the questionnaire result analysis to generate the disease prediction result, and then select the necessary test item for a more accurate diagnosis

The method may include delivering the disease prediction result and the necessary test item to a patient 10 or a medical institution (S140).

The diagnostic disease prediction and therapeutic plan recommendation system 100 of the present disclosure may deliver the test item selected through the questionnaire result analysis to the patient 10 or the medical institution for the patient to directly perform the test or the patient to perform the selected test through the medical institution.

Here, the medical institution may be a medical institution that the patient regularly visits or a medical institution that is closest to a current location of the patient.

That is, the medical institution indicates a medical institution where the patient may receive the test most quickly and conveniently.

FIG. 5 is a view showing a method of correcting the disease prediction result by reflecting a test result according to another embodiment of the present disclosure.

The disease prediction result based on the questionnaire result analysis may be less accurate in that the questionnaire result is generated based on a patient opinion. Therefore, the diagnosis may secure higher accuracy by updating the disease prediction result by reflecting the test result of the patient as follows.

The method may include receiving the test result of the necessary test item (S150). The diagnostic disease prediction and therapeutic plan recommendation system 100 of the present disclosure may receive the test result of the patient from a medical device 20 connected therewith through short-range communication or internet of things (IoT).

The method may include evaluating the disease prediction result by analyzing the received test result (S160).

The diagnostic disease prediction and therapeutic plan recommendation system 100 of the present disclosure may analyze the test result received from the medical device 20 by comparing the test result with the disease prediction result generated based on the questionnaire result.

The system 100 may determine the disease prediction result generated based on the questionnaire result analysis as a final disease prediction result when the test result matches the disease prediction result generated based on the questionnaire result analysis, and determine that the disease prediction result generated based on the questionnaire result analysis includes an error when the test result does not match the disease prediction result generated based on the questionnaire result analysis.

The method may include generating an updated disease prediction result by correcting the disease prediction result when the disease prediction result includes the error (S170).

When the disease prediction result generated based on the questionnaire result analysis includes the error, the system 100 may additionally generate a necessary questionnaire item based on the test result and provide the same to the patient again.

The system 100 may then analyze a patient answer to the additional questionnaire and generate the corrected disease prediction result by reflecting the answer and the test result.

Here, although not shown in the drawings, the method may further include: providing the patient with health coaching and counseling services based on the disease prediction result, if necessary; and predicting a possible medical cost based on the disease prediction result and the necessary test item, and providing the patient with the predicted cost. For example, when it is determined that the patient has diabetes based on the disease prediction result, the system 100 may promote long-term health enhancement of the patient by further providing the health coaching service for blood sugar control and weight control.

The present disclosure is not limited to the above-mentioned specific embodiments, and may be variously modified by those skilled in the art to which the present disclosure pertains without departing from the scope and spirit of the present disclosure as claimed in the accompanying claims. These modifications should also be understood to fall within the scope and spirit of the present disclosure as claimed in the following claims.

What is claimed is:

1. A diagnostic disease prediction and therapeutic plan recommendation system through an artificial intelligence questionnaire analysis, the system comprising:
a database (DB) storing a questionnaire scenario;
a questionnaire analysis unit configured for analyzing a questionnaire answer acquired from a patient by using an analysis artificial intelligence;
a disease prediction unit configured for generating a disease prediction result by analyzing a patient symptom through a questionnaire result systematized using a diagnosis artificial intelligence;
a test selection unit configured for selecting a necessary test item based on the disease prediction result; and
a communication unit configured for delivering the disease prediction result and the selected necessary test item to the patient or a medical institution.

2. The system of claim 1, further comprising:
a test result receiving unit linked with a medical device and configured for receiving a test result of the necessary test item;
a diagnosis determination unit configured for analyzing the test result and reviewing the disease prediction result; and
a diagnosis result correction unit configured for generating a corrected disease prediction result by reflecting the test result when the disease prediction result includes an error.

3. The system of claim 2, further comprising a diagnosis performance enhancement unit configured for enhancing performances of the analysis artificial intelligence and the diagnosis artificial intelligence by using a performance-enhancement artificial intelligence to evaluate accuracies of the analysis artificial intelligence and the diagnosis artificial intelligence with reference to one or more of the test result and electronic medical record (EMR) data of the patient and provide continuous feedback.

4. The system of claim 1, further comprising a disease prevention and management unit configured for providing the patient with health coaching and health counseling based on the disease prediction result.

5. The system of claim 1, further comprising a medical cost prediction unit configured for predicting a possible medical cost based on the disease prediction result and the necessary test item, and providing the patient with the predicted cost.

6. A diagnostic disease prediction and therapeutic plan recommendation method through an artificial intelligence questionnaire analysis, the method comprising:
systematizing questionnaire answers input by a patient by using an analysis artificial intelligence;
generating a disease prediction result by using a diagnosis artificial intelligence to identify a patient condition based on a questionnaire result and predict a disease that the patient is highly likely to have;
selecting a necessary test item based on the disease prediction result; and
delivering the disease prediction result and the necessary test item to the patient or a medical institution.

7. The method of claim 6, further comprising:
receiving a test result of the necessary test item from a linked medical device;
evaluating the disease prediction result by analyzing the test result; and
generating a corrected disease prediction result by correcting the disease prediction result when the disease prediction result includes an error.

8. The method of claim 7, further comprising enhancing accuracies of the analysis artificial intelligence and the diagnosis artificial intelligence by using a performance-enhancement artificial intelligence to evaluate performances of the analysis artificial intelligence and the diagnosis artificial intelligence with reference to one or more of the test result and electronic medical record (EMR) data of the patient and provide continuous feedback.

9. The method of claim 6, further comprising providing the patient with health coaching and health counseling based on the disease prediction result.

10. The method of claim 6, further comprising predicting a possible medical cost based on the disease prediction result and the necessary test item, and providing the patient with the predicted cost.
